(19) United States
(12) Patent Application Publication
(10) Pub. No.: US 2002/0087118 A1
(43) Pub. Date: Jul. 4, 2002

(54) Pharmaceutical delivery system

(75) Inventors: David L, Reynolds, Bromont (CA); Daniel H. MacDonald, Bromont (CA)

Correspondence Address: Mr. Roland H. Joachim, c/o Ridout & Maybee, Suite 2400, One Queen Street East, Toronto, ON, M5C 3B1, CA

(73) Assignee: Duoject Medical Systems Inc.

(21) Appl. No.: 09/750,086

(22) Filed: Dec. 29, 2000

(51) Int. Cl. A61M 37/00

(52) U.S. Cl. 604/82

(57) ABSTRACT

A pharmaceutical delivery system for reconstituting and delivering a two-part pharmaceutical composition through a catheter, consisting of a first container, typically a syringe, for containing a first component of a pharmaceutical, with a first broachable closure closing the container, and a fluid displacement apparatus configured to move fluid into and out of the container through the broachable closure; a second container containing a second component of the pharmaceutical, with a second broachable closure closing the container; a body comprising first, second and third open-ended vessels extending from the diverter valve operative to alternatively connect the first and second vessels or the first and third vessels; the first vessel communicating with a socket for receiving at least a part of the first container, including the broachable closure of the first container, the socket containing a first closure broaching member such as a cannula; the second vessel communicating with a socket for receiving at least a part of the second container, including the broachable closure of the second container; the socket containing a second closure broaching member such as a cannula. The third vessel is connected to a tubulation for delivery of the reconstituted pharmaceutical, which tubulation may, in one embodiment, also be used for filling the first container.

### FIELD OF THE INVENTION

This invention relates to the delivery of pharmaceuticals which must be packaged as two components for admixture prior to use, usually because of limited stability of the components once combined.

### BACKGROUND OF THE INVENTION

In International Published Patent Application No. WO 97/25015, there is disclosed a system for reconstituting and delivering such a two-component pharmaceutical, in which a component stored in a pharmaceutical vial and a diluent solvent or carrier stored in a protosyringe are combined through an adaptor containing a needle-bearing hub, into sockets in the opposite ends of which the vial and the protosyringe are plugged. After the components have been reconstituted and withdrawn into the protosyringe, the latter is removed from the adaptor in a manner such that an exchange of components with the hub converts the prototype into a syringe, presenting a luer ready to receive a needle or other injection instrumentality. Other arrangements have also been proposed in which containers for components cooperate with opposite ends of an adaptor which, on activation, transfer their contents to one of the containers which is then removed for use.

### SUMMARY OF THE INVENTION

The present invention is a system from which the prepared pharmaceutical can be infused through a catheter or other tubulation after admixture, without disassembly of the system.

Accordingly, the invention provides a pharmaceutical delivery system comprising a first container for receiving a first component of a pharmaceutical, a first broachable closure closing the container, and further comprising fluid displacement apparatus configured to move fluid into and out of the container through the broachable closue; a second container containing a second component of the pharmaceutical, and a second broachable closure closing the container; a body comprising a diverter valve and first, second and third vessels extending to open ends from the diverter valve, which valve is operative to alternatively connect the first and second vessels or the first and third vessels; a first socket communicating with the open end of the first vessel and configured to receive at least a part of the first container, including the broachable closure of the first container, the first socket containing a first closure broaching member; a second socket communicating with the open end of the second vessel and configured to receive at least a part of the second container, including the broachable closure of the second container; the second socket containing a second closure broaching member; and a tubulation for delivery of the pharmaceutical extending from the open end of the third vessel. An example of closure broaching member is a cannula.

Further features of the invention will become apparent from the following description of a preferred embodiment thereof.

### SHORT DESCRIPTION OF THE DRAWINGS

FIG. 1 is a view of a first embodiment of a system in accordance with the invention, prior to preparation for use;

FIG. 2 is a corresponding exploded view of components of the system;

FIGS. 3-7 are corresponding views, each to some extent fragmentary, illustrating subsequent stages of activation of the system to ready it for actual use;

FIGS. 8-12 are views, each to some extent fragmentary, of a second embodiment of the invention, illustrating stages in the activation of the system: and

FIGS. 13-17 illustrate certain components of a further embodiment of the invention, and certain stages in its activation.

### DESCRIPTION OF THE PREFERRED EMBODIMENT

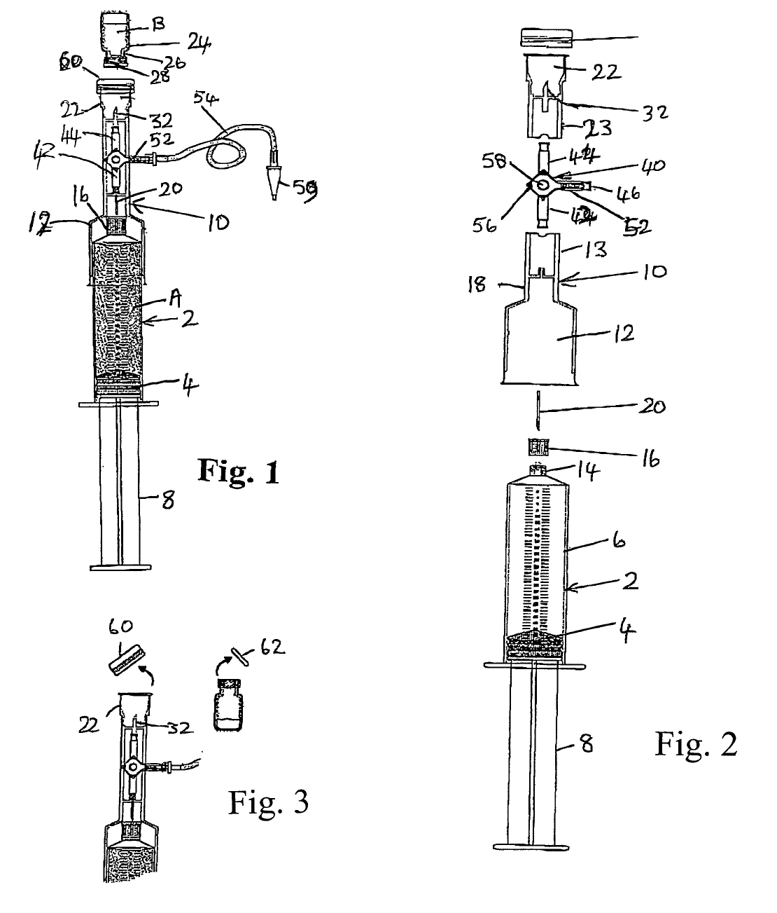

Referring to FIGS. 1 and 2, the system comprises a first container in the form of a syringe 2, having a fluid displacement apparatus. In the illustrated embodiment, the fluid displacement apparatus is in the form of a piston 4 movable axially in sealing engagement with an inside wall of a syringe body 6, the piston being reciprocable within the syringe body by a plunger 8. The syringe shown, which has a molded plastic body, is exemplary only, and another type of syringe or container capable of expelling or aspirating fluid may be utilized provided that it has appropriate fluid displacement apparatus, and provides a container for a component of a pharmaceutical which has a broachable closure which is broachable when it is forced onto the first closure broaching member 20 in a first socket attached to a first vessel defined by a body of the system. An alternative example of such a container is an ampoule having a frangible seal at its delivery end that can be broken by the broaching means.

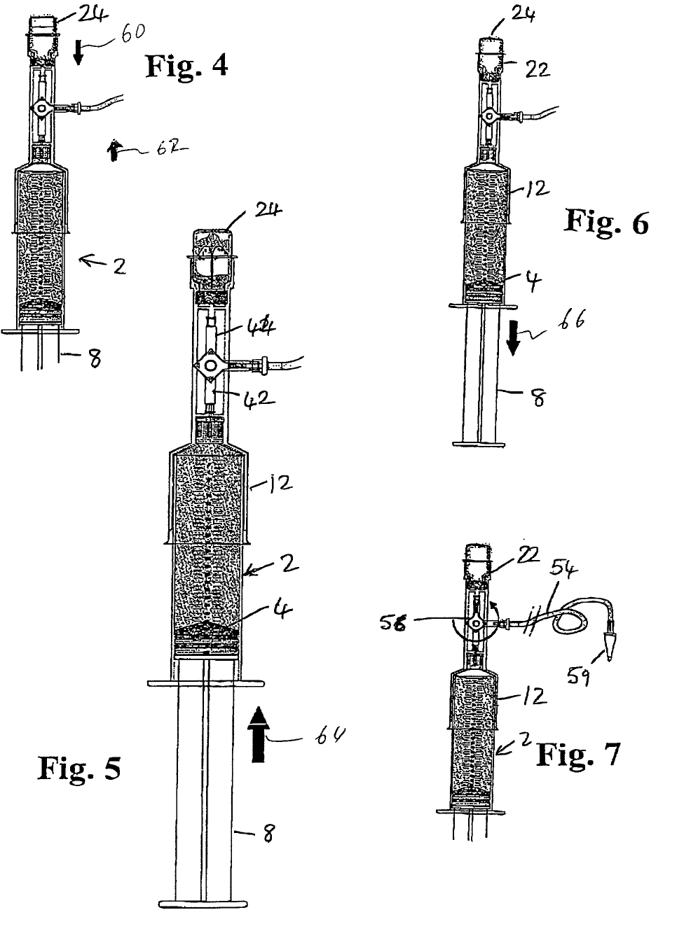

In the embodiment described, the body 10 defines in part a first vessel 42 having a first socket 12 communicating with and extending from an end thereof which receives an end of the syringe 2 equipped with a luer 14 (see FIG. 3) provided with a closure in the form of a rubber cap 16. On activation of the syringe, the cap 16 is forced into a narrowed portion 18 of the socket such that a needle or cannula 20 penetrates the cap (see FIG. 4). Other broaching means could be used; for example, the closure could be provided by a plug axially displaceable within a neck of the syringe so as to open a passageway, the plug being displaced by broaching means in the form of a rod replacing the needle.

The first vessel 42 is only one of three vessels contained within or defined by the body 10. A second vessel 44 communicates at one end with a second socket 22 extending from the vessel 44. The second socket is configured to receive at least part of the second container, typically a pharmaceutical vial 24, including a neck 26 and closure 28 of the vial. On actuation, the neck 26 of the vial is forced into a narrowed portion 30 of the socket 21 so that the closure 28 is broached by a cannula 32. As in the case of the first container, alternative closures and broaching means are possible. The vial contains a second component of the pharmaceutical. Typically, the syringe will contain a first component in the form of a liquid solvent, diluent or suspension medium for a liquid or solid second component in the vial but other arrangements are possible, provided that at least one component is liquid.

In the embodiment shown, the sockets 12 and 22 have tubular extensions, 13 and 23 respectively, the tubular extensions being welded together so as to enclose the first and second vessels 42 and 44 which extend from a valve assembly 40, and support a third vessel 46 of the assembly which projects through a side wall 48 of a chamber 50 formed by cooperation of the extensions 13 and 23. Open ends of the vessels 42 and 44 communicate with the sockets 12 and 22 through the cannulas 20 and 32. The vessel 46 projects through the side, and its open end 52 receives a flexible tubulation 54 through which a reconstituted pharmaceutical may be dispensed from the system, for example, to a connector 59 which may be coupled to a nozzle (not shown) to provide a catheter for introduction into a body orifice. It will be noted that the tubular extensions are welded together so that the sockets 12 and 22 are coaxial, thus providing a strong structure, and facilitating proper relative orientation of the syringe 2 and vial 24 during reconstitution of the pharmaceutical.

Also projecting through the wall 48 is an actuator lever 56 which moves a valve member 58 between a first position and a second position. The first position forms a passage, defined by the valve member, establishing communication between the first vessel 42, and the second vessel 44. The second position forms a passage defined by the valve member establishing communication between the first vessel and the open end 52 of the third vessel 46.

The open end of the socket 22 is closed by a cap 60 prior to insertion of the vial 24, while engagement of the cap 16 with the entrance to the narrowed portion 18 of the first vessel closes off that portion. The penetrable closure 28 of the vial 24 is initially closed in conventional fashion by a flip-off cap 62.

In use, starting with the system shown in FIG. 1, it is checked that the lever 56 is set so that the valve member is in the first position permitting fluid communication between the first and second vessels (42 and 44). The caps 60 and 62 are flipped off as shown in FIG. 3. As shown in FIG. 4, the vial 24 is then pressed into the second socket 21 as shown in FIG. 4 such that the neck 26 of the vial is forced into the portion of the socket so that the closure 28 is penetrated by the cannula 32. The syringe is pressed in the direction of arrow 62, into the socket 12. The cap 16 is forced into a narrowed portion of the socket causing the cannula 20 to penetrate the cap 16.

At this point, the first vessel and the syringe 2 are in fluid communication with the second vessel 22 and the vial 24. Then, the plunger 8 is actuated so as to eject fluid through the valve member 58 into the vial 24 (see FIG. 5). If the syringe contents A are a liquid, the transferred liquid is swilled in the vial to dissolve, suspend or dilute the content B of the vial, and the resulting liquid is aspirated back into the syringe by manipulation of the plunger 8 (FIG. 6) in the direction of arrow 66. If only the content of the vial is liquid, then the plunger may be used to force gas from the syringe into the vial which is used to aspirate liquid from the vial back into the syringe to dissolve or suspend the content of the latter. In either case, after aspiration, the assembly may then be inverted several times to complete dissolution, admixture or suspension. It should be noted that no additional vents or the like are required in or between the syringe 2 and the vial 24, the sole communication being through the vessels 42 and 44.

The lever 56, as shown in FIG. 7, is moved to reposition the valve member to a position in which it connects the first 12 and second 22 vessels, which enables the syringe to be utilized to deliver the content of the latter into the catheter formed by tubulation 54. The syringe plunger may be actuated manually, or with the assistance of a syringe actuator connected to or in place of the plunger 8.

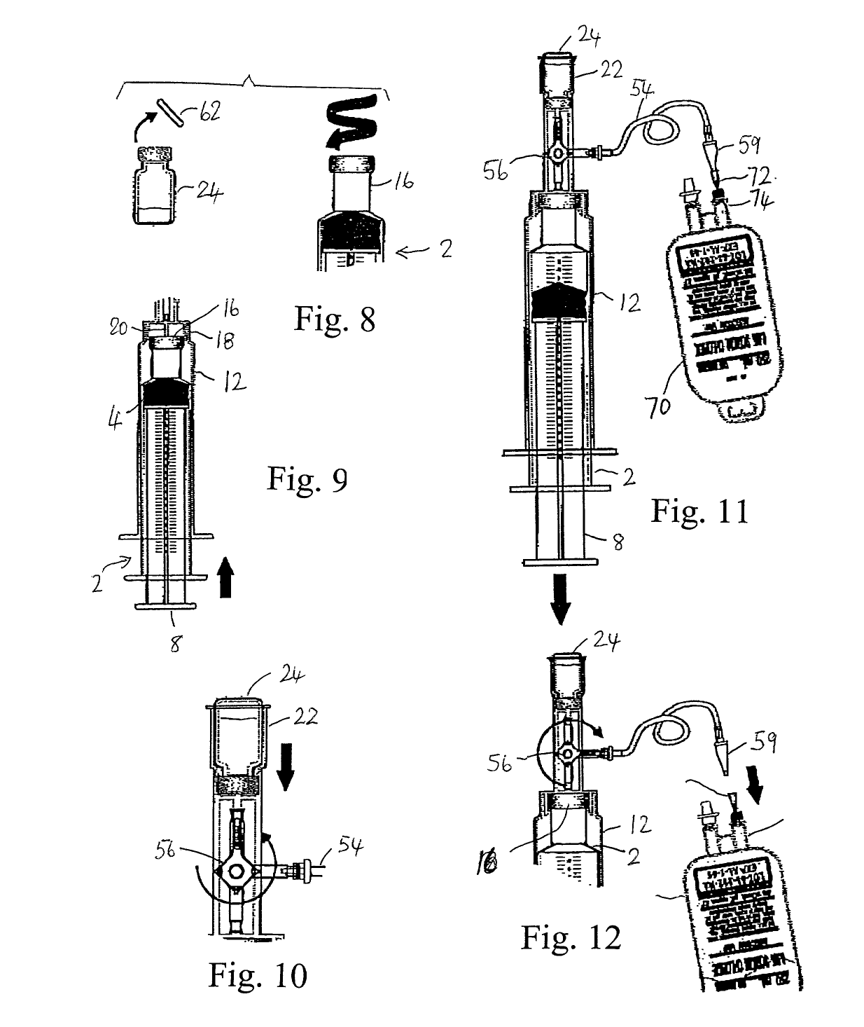

Referring now to FIGS. 8-15, the apparatus shown is essentially similar to that of the preceding figures, and the same reference numerals are utilized to identify the same or functionally similar parts. The cap 16 and narrowed portion 18 are somewhat larger in diameter, and an additional component is shown in the form of a plastic bag 70 of the type widely used for the intravenous (I.V.) administration of water or saline solutions, and a needle 72 of standard type commonly used with syringes.

The syringe 2, unlike that of the previous embodiment, is supplied empty, and the system of the invention is used for filling this syringe from the I.V. bag 70, as well as for delivering the reconstituted pharmaceutical, according to the procedure described below.

The empty syringe 2 with its plunger 8 located so that the piston 4 is adjacent the luer 14, is fully inserted in the socket 12 so that a penetrable diaphragm of the cap 16 is broached (see FIG. 9), and the vial 4 is likewise fully inserted into the socket 22. The valve member 58 is moved to its second position, establishing communication between the syringe 2 and the tubulation 54. The needle 72 is applied to the adaptor 59, and inserted into a nozzle 74 of the bag. The plunger 8 is then withdrawn with the piston 4 so as to aspirate liquid, such as sterile water or saline solution, from the bag 70 into the syringe 2. The valve member 58 is then moved to its first position, and the needle and bag are removed from the adaptor 59, from which point reconstitution proceeds as already described with reference to FIGS. 5 to 7.

An advantage of this embodiment is that it permits utilization of economical containers of diluent fluid which are already readily available and fully certified.

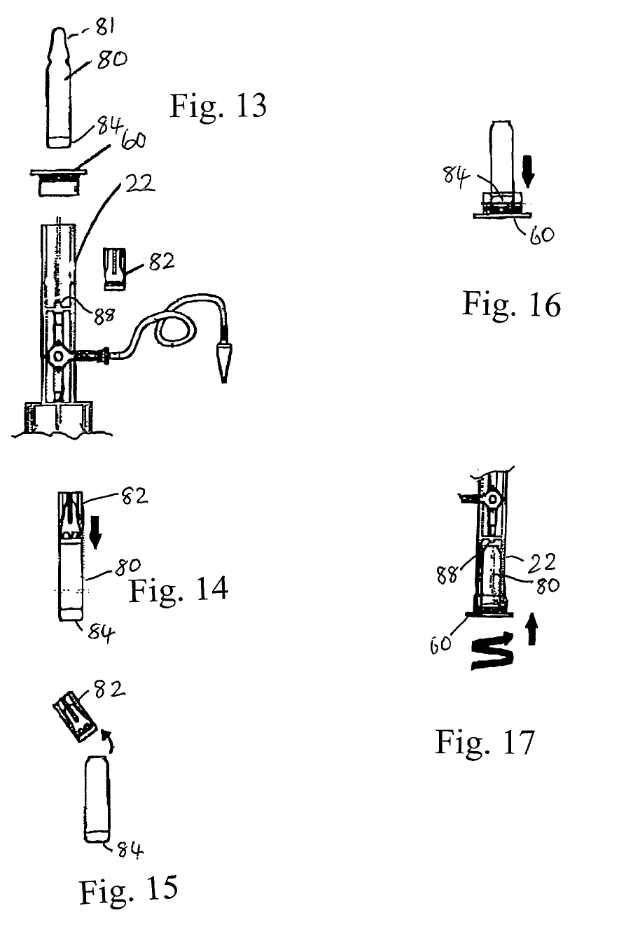

In a further modification of the invention illustrated in FIGS. 13 to 17, the vial 24 is replaced by a glass ampoule 80. An ampoule breaker 82 is provided, and the socket 22 is made deep enough to house the body of the ampoule. The cap 60 is adapted to screw into the exterior wall of the socket 22.

In use, the breaker 82 is used to break off the sealed tip of the ampoule (FIGS. 14 and 15), and the base 84 of the ampoule is inserted in the cap 60 (FIG. 16) which is then screwed onto the socket 22 (FIG. 17) so as to bring the broken neck 86 of the ampoule against a nipple 88 which replaces the cannula 32. Reconstitution then proceeds as before.

It will be appreciated that the above described embodiments are exemplary only, and that variations and modifications are possible within the scope of the appended claims.

I claim:

1. A pharmaceutical delivery system comprising:
a first container for receiving a first component of a pharmaceutical, a first broachable closure closing the container, the first container further comprising fluid displacement means for moving fluid into and out of the container through the broachable closure;
a second container containing a second component of the pharmaceutical, and a second broachable closure closing the container;
a body comprising a diverter valve and first, second and third vessels extending to open ends from the diverter valve which valve is operative to alternatively connect the first and second vessels or the first and third vessels;
a first socket communicating with the open end of the first vessel, for receiving at least a part of the first container, including the broachable closure of the first container, the socket containing first broaching means to broach that closure;
a second socket communicating with an open end of the first vessel, for receiving at least a part of the second container, including the broachable closure of the second container; the socket containing second broaching means to broach that closure; and
a tubulation for delivery of the pharmaceutical connected to the open end of the third vessel.

2. A system according to claim 1, wherein at least one of the broachable closures is a penetrable seal, and the associated broaching means is a cannula.

3. A system according to claim 1, wherein the first container is a syringe.

4. A system according to claim 3, wherein the syringe has a luer, and the penetrable closure is a rubber cap over the luer.

5. A system according to claim 1, wherein the second container is a pharmaceutical vial.

6. A system according to claim 1, wherein both the first and second sockets have narrowed portions connecting them to the first and second vessels, the narrowed portions containing broaching means in the form of cannulas directed away from the vessels towards the sockets, and the narrowed portions are dimensioned so that portions of the first and second containers including the closures thereof are a press fit therein such that the closures of the containers may be pressed onto the cannulas to broach the closures.

7. A system according to claim 1, in which the first container is prefilled with the first component of the pharmaceutical.

8. A system according to claim 1, in which the first container is initially empty, and the tubulation attached to the third vessel has an adaptor usable for aspiration of a liquid component of the pharmaceutical from a container as well as for delivery of the reconstituted pharmaceutical.

9. A system according to claim 1, wherein the first and second sockets are coaxial and rigidly connected to one another through a housing accommodating the diverter valve.

10. A pharmaceutical delivery system comprising:
a first container for receiving a first component of a pharmaceutical, a first broachable closure closing the container, the first container further comprising fluid displacement means for moving fluid into and out of the container through the broachable closure;
a second container containing a second component of the pharmaceutical, and a second broachable closure closing the container;
a body comprising a diverter valve and first, second and third vessels extending to open ends from the diverter valve which valve is operative to alternatively connect the first and second vessels or the first and third vessels;
a first socket communicating with the open end of the first vessel, for receiving at least a part of the first container, including the broachable closure of the first container, the socket containing first broaching means to broach that closure;
a second socket communicating with an open end of the first vessel, for receiving at least a part of the second container, including the broached closure of the second container; the socket containing means to establish communication with the content of the second container; and
a tubulation for delivery of the pharmaceutical connected to the open end of the third vessel.

11. A system according to claim 10, wherein the second container is an ampoule having a frangible neck portion and broachable by breaking off of said neck portion.
